(19) United States
(12) Patent Application Publication
(10) Pub. No.: US 2005/0002919 A1
(43) Pub. Date: Jan. 6, 2005

(54) Cadherin directed molecular and cellular localization

(75) Inventors: Michael B. Brenner, Newton, MA (US); David M. Lee, Needham, MA (US)

Correspondence Address: Maria A. Trevisan;Wolf, Greenfield & Sacks, P.C., 600 Atlantic Avenue, Boston, MA, 02210, US

(73) Assignee: The Brigham and Women's Hospital, Inc., Boston, MA (US)

(21) Appl. No.: 10/864,087

(22) Filed: Jun. 9, 2004

Related U.S. Application Data
(60) Provisional application No. 60/477,170, filed on Jun. 9, 2003.

(51) Int. Cl. A61K 48/00

(52) U.S. Cl. 424/93.21

(57) ABSTRACT

Methods for treating disease by administering a genetically modified cadherin-expressing cell are provided.

### RELATED APPLICATIONS

This application claims priority to U.S. Provisional Patent Application filed Jun. 9, 2003, entitled “CADHERIN DIRECTED MOLECULAR AND CELLULAR LOCALIZATION”, Ser. No. 60/477,170, the contents of which are incorporated by reference herein in their entirety.

### GOVERNMENT SUPPORT

This invention was made in part with government support under grant number 1RO1-AR48114-01 from the National Institutes of Health. The Government may retain certain rights in the invention.

### FIELD OF THE INVENTION

This invention provides methods and compositions for targeting cadherin-expressing cells to particular locations in the body of a subject.

### BACKGROUND OF THE INVENTION

The adhesive interactions between cells and between cells and the extracellular matrix are believed to play critical roles in a wide variety of processes including, for example, modulation of the immune system, regulation of developmental processes and tumor progression and metastasis. These interactions are mediated by adhesion molecules which transduce information from the extracellular environment to the cell.

Four families of adhesion molecules that mediate these interactions have been identified: the integrins, the cadherins, the selecting, and immunoglobulin-related molecules. In general, adhesion molecules are transmembrane proteins that contain an extracellular domain for interacting with an extracellular matrix or cellular component, a transmembrane domain spanning the cell membrane and a cytoplasmic domain for interacting with one or more cytoskeletal or cytoplasmic components.

The cadherins play an important role in the establishment and maintenance of intercellular connections between cells of the same type (reviewed in Geiger B. et al. (1992) Annual Review of Cell Biology 8:307; Kemler R. (1993) Trends in Gastroenterology 9:317; Takeichi M. (1990) Annual Review of Biochem. 59:237; Takeichi M. (1991) Science 251:1451). Cadherins are a superfamily of structurally related molecules that function in Ca⁺²-dependent homophilic adhesion. Cadherins are expressed on cells that form solid tissues, and are responsible for establishing cell polarity, and maintaining tissue morphology.

The cadherins are synthesized as precursors that are cleaved during post-translational processing. The mature cadherins are single chain molecules that include a relatively large extracellular domain (typically divided into five sections or “ectodomains”), a single transmembrane region and a cytoplasmic tail. Among the classical cadherins (i.e., P- (placenta), E- (epithelial), and N- (neural) cadherin), the cytoplasmic domain contains the highest degree of homology. The high degree of homology observed for the cytoplasmic domain reportedly is a reflection of the association of cadherins with a group of intracellular proteins, called catenins, that stabilize cadherin active conformation (Kemler R. (1993) Trends in Gastroenterology 9:317). It is generally believed that sequences in the extracellular domain are necessary to mediate homophilic (i.e., cadherin-to-cadherin) binding and heterophilic binding.

### SUMMARY OF THE INVENTION

The invention is based, in part, on the discovery that cadherin polypeptides can be used to target cells to specific cells, tissues or locations in the body. Methods and compositions provided by the invention further provide for the modulation of cellular functions in cells and tissues that express cadherins. Thus these functions can be targeted by the compositions of the invention. In addition, the invention provides isolated cells that have been genetically modified to express, on their cell surface, molecules that bind to ligands present on the surface of target cells. Preferably, these ligands are themselves cadherins, and the cells are genetically modified to express at least one cadherin that they do not naturally express. The genetically modified cells specifically adhere to their target cell type in vitro and in vivo. Methods for delivering the modified cells to target tissues and methods for using the modified cells to deliver pharmaceutical agents, diagnostic agents and/or toxins also are provided.

Thus in one aspect, the invention provides a method for delivering cells to a target tissue of a subject, comprising administering an isolated cell with genetically altered cadherin expression to a subject in need thereof, wherein the genetically altered cadherin expression allows the isolated cell to bind to a target tissue.

In another aspect, the isolated cell is altered to express a cadherin on its cell surface via nongenetic methods (e.g., ionic attachment of a cadherin on a cell surface).

There are several embodiments that apply to this and other aspects of the invention and these are recited below.

The genetically altered cadherin expression may be a decrease or an increase in the expression of an endogenously expressed cadherin. The genetically altered cadherin expression may also be expression of a cadherin not endogenously expressed by the isolated cell. In some embodiments, the genetically altered cadherin expression is lack of expression of an endogenously expressed cadherin. The genetically altered cadherin expression may include altered expression of two or more cadherins.

In some embodiments, the genetically altered cadherin expression is altered expression of a classical or non-classical cadherin. A classical cadherin may be selected from the group consisting of E- (epithelial) cadherin, N- (neural) cadherin, P- (placental) cadherin, VE- (vascular endothelial) cadherin, R- (retinal) cadherin, M-cadherin, and C-cadherin. A non-classical cadherin may be selected from the group consisting of cadherin-6 (K-cadherin), cadherin-7, cadherin-8, cadherin-11 (OB-cadherin), cadherin-12 (Br-cadherin), cadherin-13 (T- (truncated) cadherin or H- (heart) cadherin), cadherin-14, cadherin-15 (M-cadherin), PB-cadherin, LI-cadherin, T-cadherin, protocadherins (e.g., protocadherin-42, protocadherin-43, protocadherin-68), desmocollins (e.g., desmocollin-1, desmocollin-2, desmocollin-3, desmocollin-4), desmogleins (e.g., desmoglein-1, desmoglein-2), and cadherin-related neuronal receptors. The cadherin may also be a cadherin-fusion protein, such as but not limited to a cadherin-Fc fusion protein.

Preferably, the cadherin is one known to bind to a tissue selected from the group consisting of synovium, blood vessel, lung, bone marrow, bone, colon, kidney, epidermis, joint, brain, neuronal, muscle, pancreas, liver, heart tissue, and pancreas.

The genetically altered cadherin expression is expression of a cadherin full length polypeptide or a fragment thereof, according to some embodiments. In others it is the genetically altered cadherin expression is expression of a cadherin mutant or a functionally equivalent cadherin variant.

The isolated cell may be a progenitor cell such as a stem cell or a precursor. The isolated cell may be a stem cell, such as a totipotent stem cell or a pluripotent stem cell. The totipotent stem cell may be an embryonic stem cell or an adult stem cell. Preferably the cells are of human origin. The pluripotent stem cell may be tissue-specific but is not so limited. The pluripotent stem cell may be a neural stem cell, a skin stem cell, a liver stem cell, a pancreatic β islet cell, a mesenchymal stem cell, and a hematopoietic stem cell, but it is not so limited. In other embodiments, the isolated cell is a multipotent or unipotent precursor cell.

In still other embodiments, the isolated cell is a terminally mature cell, such as but not limited to a bone marrow stromal cell, a hepatocyte, a synovial cell, a muscle cell, a cardiac cell, a neural cell, and a skin cell.

The isolated cell may be a primary cell. In some embodiments, the primary cell is harvested from the subject. The primary cell may be harvested from a different tissue than the target tissue. The primary cell may be cultured in vitro prior to or after genetic alteration. In some embodiments, the isolated cell is derived from a cell line. In important embodiments, the isolated cell has proliferative activity.

In some embodiments, the isolated cell is administered to the subject by parenteral administration, although it is not so limited. The isolated cell may be administered to the subject by intravenous, intraperitoneal, subcutaneous, intramuscular or intratissue administration.

In one embodiment, the target tissue is abnormal. In a related embodiment, the subject has or is at risk of developing muscular dystrophy, cirrhosis, a hematopoietic abnormality, Alzheimer's disease, Parkinson's disease, cystic fibrosis, arthritis, nephritis, vasculitis, asthma, autoimmune hepatitis, fibrosis, stroke, diabetes, cardiac infarction, hepatic failure, multiple sclerosis, or cancer. The cadherin or combination of cadherins to be used in each of these situations will depend upon the tissue affected. In some embodiments, the cadherin is N-cadherin and the target tissue is neural tissue. In other embodiments, the cadherin is M-cadherin and the target tissue is muscle. In still other embodiments, the cadherin is E-cadherin and the target tissue is liver or skin.

In some embodiments, the isolated cell has genetically altered reporter marker expression. The reporter marker may selected from the group consisting of luciferase, green fluorescent protein (GFP), EGFP and β galactosidase. In still other embodiments, the isolated cell has genetically altered selection marker expression. The selection marker may be selected from the group consisting of neomycin (G418), hygromycin, bleomycin, phleomycin and puromycin, although it is not so limited.

The genetically altered cadherin expression may be stable or transient.

In yet another aspect, the invention provides a composition comprising an isolated non-experimental cell having genetically altered cadherin expression. In some embodiments, the composition comprises a pharmaceutically acceptable carrier. It may be further formulated for in vivo administration. In some important embodiments, the cell is a non-transformed cell.

In yet another aspect, the invention provides a cell comprising a progenitor cell, and preferably a human progenitor cell, genetically altered to express a cadherin. In certain embodiments, the cadherin is selected from the group consisting of E-cadherin, N-cadherin, P-cadherin, VE-cadherin, vascular cadherin, R-cadherin, C-cadherin cadherin-4, cadherin-6 (K-cadherin), cadherin-7, cadherin-8, cadherin-9, cadherin-10, cadherin-11 (OB-cadherin), cadherin-12 (Br-cadherin), cadherin-13, cadherin-14, cadherin-15 (M-cadherin), cadherin-19, cadherin-20, PB-cadherin, ksp-cadherin, LI-cadherin, protocadherin 42, protocadherin-43, protocadherin-68, protocadherin alpha 1, protocadherin beta 15, protocadherin gamma A1, protocadherin gamma B1, protocadherin gamma C3, PCDH7 (BH-Pcdh)a, protocadherin (PCDH8), protocadherin-Xa, OL-protocadherin, desmocollin, desmocollin-1, desmocollin-2, desmocollin-3, desmocollin-4, desmoglein, desmoglein-1, desmoglein-2, and cadherin-related neuronal receptors.

Different embodiments of the progenitor cell are discussed above and apply equally to this latter aspect of the invention.

These and other aspects of the invention, as well as various advantages and utilities, will be more apparent with reference to the detailed description of the preferred embodiments and to the accompanying drawings.

### BRIEF DESCRIPTION OF THE DRAWINGS

The Examples refer to and include a brief description of various figures. It is to be understood that the drawings or figures are illustrative only and are not required for the enablement of the inventions disclosed herein.

FIG. 1 is a diagram of a pCEP4 vector encoding a cadherin and IgG-Fc domain driven by a pCMV promoter and having a hygromycin resistance selectable marker.

FIG. 2 is a diagram of a pcDNA3 vector encoding a mouse cadherin-11 under the control of a pCMV promoter and having a luciferase reporter coding sequence and a neomycin resistance selectable marker.

FIG. 3 is a diagram of a pMIEV vector encoding mouse cadherin-11 under the control of an LTR and having an EGFP reporter coding sequence (referred to as mouse cadherin-11-pMIEV).

FIG. 4 is a diagram of a pMIEV vector encoding mouse E-cadherin under the control of an LTR and having an EGFP reporter coding sequence (referred to as mouse E-cadherin-pMIEV).

FIG. 5 is a diagram of a pcDNA3 vector encoding a mouse cadherin-11 under the control of a pCMV promoter and having a EGFP reporter coding sequence and a neomycin resistance selectable marker.

### DETAILED DESCRIPTION OF THE INVENTION

The invention is premised, in part, on the discovery that cadherin molecules can be used to target delivery of cells to specific cells, tissues or locations in a subject. Cadherin molecules are differentially expressed both temporally and spatially in cells and tissues, and this differential expression can be used to specifically deliver cells to various locations throughout the body of a subject. Temporally and spatially regulated expression of cadherins provides an opportunity to target therapies designed to ameliorate disorders in particular cells, tissues, or locations in the body.

Cadherins are transmembrane molecules that, inter alia, mediate binding of cells to each other through homophilic and heterophilic interactions. The cadherins are proposed to mediate adhesion of like cells to each other, as well as adhesion of cells of different lineages to each other. The present invention is based in part on the ability of cadherins to mediate adhesion between cells of the same or different lineage.

The cells of the invention are genetically modified to alter cadherin expression. Cadherin expression refers to the expression of a cadherin nucleic acid or a cadherin polypeptide. Cell surface expression refers to the expression of a cadherin polypeptide (or fragment thereof) at the surface of a cell. Preferably, the cells of the invention are genetically modified to express cadherin polypeptides (or fragments thereof) at the cell surface.

Cadherin molecules therefore include cadherin nucleic acids and cadherin polypeptides, and mutants, variants (including functionally equivalent variants), and fragments thereof, provided they are capable of being expressed on the surface of a cell for interaction with a binding partner, including as part of a fusion protein. The cadherin nucleic acid and cadherin polypeptides can be full length, but are not so limited. Mutants, variants and fragments of cadherins retain the ability to bind to a cadherin receptor (e.g., a cadherin) on a target cell or tissue.

The cadherins can be classical cadherins or non-classical cadherins. Classical (type I) cadherins are cadherins that comprise the histidine-alanine-valine (HAV) cell adhesion recognition (CAR) sequence. These cadherins regulate, inter alia, epithelial, endothelial, neural and cancer cell adhesion. They have a similar structure comprised of five extracellular domains, a single hydrophobic domain and two cytoplasmic domains. Calcium binding motifs are located in the extracellular domains. Classical cadherins include E- (epithelial) cadherin, N- (neural) cadherin, P- (placental) cadherin, VE- (vascular endothelial) cadherin, R- (retinal) cadherin, M-cadherin, and C-cadherin. Non-classical (types II-X) cadherins are cadherins that contain calcium binding motifs in the extracellular domains but do not have the HAV CAR. Non-classical cadherins include cadherin-6 (K-cadherin), cadherin-7, cadherin-8, cadherin-11 (OB-cadherin), cadherin-12 (Br-cadherin), cadherin-13 (T- (truncated) cadherin or H- (heart) cadherin), cadherin-14, cadherin-15 (M-cadherin), PB-cadherin, LI-cadherin, T-cadherin, protocadherins (e.g., protocadherin-42, protocadherin-43, protocadherin-68), desmocollins (e.g., desmocollin-1, desmocollin-2, desmocollin-3, desmocollin-4), desmogleins (e.g., desmoglein-1, desmoglein-2), and cadherin-related neuronal receptors. Cadherins as a group include but are not limited to E-cadherin, N-cadherin, P-cadherin, VE-cadherin, vascular cadherin, cadherin-4, cadherin-6, cadherin-7, cadherin-8, cadherin-9, cadherin-10, cadherin-11 (OB-cadherin), cadherin-12, cadherin-13, cadherin-14, cadherin-15, cadherin-19, cadherin-20, ksp-cadherin, LI-cadherin, protocadherin 42, protocadherin alpha 1, protocadherin beta 15, protocadherin gamma A1, protocadherin gamma B1, protocadherin gamma C3, PCDH7 (BH-Pcdh)a, protocadherin (PCDH8), protocadherin-Xa, OL-protocadherin, and protocadherin 68.

A cadherin polypeptide is a polypeptide that can be expressed in a cell and presented on a cell surface, the cell surface portion being a portion of a cadherin capable of binding selectively to a cadherin ligand (e.g., a cadherin) on the surface of a cell. Cadherin polypeptides include polypeptides having amino acid sequences of the following GenBank Accession Numbers:
Homo sapiens E-cadherin, Genbank Acc. No.: CAA78353.1;
Homo sapiens N-cadherin, Genbank Acc. No.: AAB22854.1;
Homo sapiens P-cadherin, Genbank Acc. No.: CAA45177.1;
Homo sapiens cadherin-4, Genbank Acc. No.: AAA35627.1;
Homo sapiens VE-cadherin, Genbank Acc. No.: CAA56306.1;
Homo sapiens cadherin-6, Genbank Acc. No.: BAA06562.1;
Homo sapiens cadherin-7, Genbank Acc. No.: CAC13127.1;
Homo sapiens cadherin-8, Genbank Acc. No.: AAA35628.1;
Homo sapiens cadherin-9, Genbank Acc. No.: BAA87416.1;
Homo sapiens cadherin-10, Genbank Acc. No.: AAD44017.1;
Homo sapiens cadherin-11, Genbank Acc. No.: AAA35622.1;
Homo sapiens cadherin-12, Genbank Acc. No.: AAA35623.1;
Homo sapiens cadherin-13, Genbank Acc. No.: AAA35624.1;
Homo sapiens cadherin-15, Genbank Acc. No.: BAA12012.1;
Homo sapiens Ksp-cadherin (CDH16), Genbank Acc. No.: AAC34255.1;
Homo sapiens LI-cadherin, Genbank Acc. No.: CAA58231.1;
Homo sapiens cadherin-14, Genbank Acc. No.: AAB02933.1;
Homo sapiens cadherin-19 (CDH19), Genbank Acc. No.: CAC13126.1;
Homo sapiens cadherin-20, Genbank Acc. No.: AAG23739.1;
Homo sapiens protocadherin Alpha 1 (PCDH-alpha1), Genbank Acc. No.: AAD43699.1;
Homo sapiens protocadherin beta 1 (PCDH-beta1), Genbank Acc. No.: AAD43749.1;
Homo sapiens protocadherin beta 15 (PCDH-beta15), Genbank Acc. No.: AAD43755.1;
Homo sapiens protocadherin gamma A1 (PCDH-gamma-A1), Genbank Acc. No.: AAD43712.1;
Homo sapiens protocadherin gamma B1 (PCDH-gamma-B1), Genbank Acc. No.: AAD43724.1;
Homo sapiens protocadherin gamma C3 (PCDH-gamma-C3), Genbank Acc. No.: AAD43731.1;
Homo sapiens protocadherin 42 (PC42), Genbank Acc. No.: AAA36419.1;
Mus musculus vascular cadherin-2, Genbank Acc. No.: CAA69965.1;
Homo sapiens PCDH7 (BH-Pcdh)a, Genbank Acc. No.: BAA25194.1;
Homo sapiens protocadherin (PCDH8), Genbank Acc. No.: AAC70009.2;
Homo sapiens PCDH-XE, protocadherin-Xa, Genbank Acc. No.: BAA90765.1;
Mus musculus OL-protocadherin, Genbank Acc. No.: AAD00651.1; and
Homo sapiens protocadherin 68 (PCH68), Genbank Acc. No.: AAB84144.1.

A cadherin nucleic acid is a nucleic acid that encodes for and from which the cadherin polypeptides recited herein may be produced. Cadherin nucleic acids include nucleic acids having the nucleotide sequences (or degenerates thereof) of the following GenBank Accession Numbers:
Homo sapiens E-cadherin, Genbank Acc. No.: Z13009;
Homo sapiens N-cadherin, Genbank Acc. No.: S42303;
Homo sapiens P-cadherin, Genbank Acc. No.: X63629;
Homo sapiens cadherin-4, Genbank Acc. No.: L34059;
Homo sapiens VE-cadherin, Genbank Acc. No.: X79981;
Homo sapiens cadherin-6, Genbank Acc. No.: D31784;
Homo sapiens cadherin-7, Genbank Acc. No.: AJ007611;
Homo sapiens cadherin-8, Genbank Acc. No.: L34060;
Homo sapiens cadherin-9, Genbank Acc. No.: AB035302;
Homo sapiens cadherin-10, Genbank Acc. No.: AF039747;
Homo sapiens cadherin-11, Genbank Acc. No.: L34056;
Homo sapiens cadherin-12, Genbank Acc. No.: L34057;
Homo sapiens cadherin-13, Genbank Acc. No.: L34058;
Homo sapiens cadherin-15, Genbank Acc. No.: D83542;
Homo sapiens Ksp-cadherin (CDH16), Genbank Acc. No.: AF016272;
Homo sapiens LI-cadherin, Genbank Acc. No.: X83228;
Homo sapiens cadherin-14, Genbank Acc. No.: U59325;
Homo sapiens cadherin-19 (CDH19), Genbank Acc. No.: AJ007607;
Homo sapiens cadherin-20, Genbank Acc. No.: AF217289;
Homo sapiens protocadherin Alpha 1 (PCDH-alpha1), Genbank Acc. No.: AF152305;
Homo sapiens protocadherin beta 1 (PCDH-beta1), Genbank Acc. No.: AF152488;
Homo sapiens protocadherin beta 15 (PCDH-beta15), Genbank Acc. No.: AF152494;
Homo sapiens protocadherin gamma A1 (PCDH-gamma-A1), Genbank Acc. No.: AF152318;
Homo sapiens protocadherin gamma B1 (PCDH-gamma-B1), Genbank Acc. No.: AF152330;
Homo sapiens protocadherin gamma C3 (PCDH-gamma-C3), Genbank Acc. No.: AF152337;
Homo sapiens protocadherin 42 (PC42), Genbank Acc. No.: L11370;
Mus musculus vascular cadherin-2, Genbank Acc. No.: Y08715;
Homo sapiens PCDH7 (BH-Pcdh)a, Genbank Acc. No.: AB006755;
Homo sapiens protocadherin (PCDH8), Genbank Acc. No.: AF061573;
Homo sapiens PCDH-XE, protocadherin-Xa, Genbank Acc. No.: AB026187;
Mus musculus OL-protocadherin, Genbank Acc. No.: U88549; and
Homo sapiens protocadherin 68 (PCH68), Genbank Acc. No.: AF029343.

The cadherins may include one or more cadherin cell adhesion recognition (CAR) domains. A CAR domain is a domain within most cadherins that has been implicated in cadherin-mediated cell adhesion. The CAR may be a classical or non-classical CAR. An example of a classical CAR sequence is His-Ala-Val (HAV). In some embodiments, the cadherin molecules of the invention include the HAV sequence. Non-classical CAR sequences are also known in the art. Classical and non-classical CAR sequences are reported in U.S. Pat. Nos. 6,433,149; 6,358,920; 6,203,788; 6,333,307; 6,417,325; 6,465,427; 6,358,920; 6,433,149; 6,203,788; 6,346,512; 6,451,318; 6,451,971; 6,458,939; 6,447,776; 6,465,427; 6,417,325; 6,433,149; 6,277,824; 6,472,368; and 6,472,367; and published U.S. patent application Ser. Nos. 20020146687; 20020151475; 20020146687; 20020169106; and 20020123044. Examples of CAR included in U.S. Pat. No. 6,433,149 include DDK, IDDK (SEQ ID NO:1), DDKS (SEQ ID NO:2), VIDDK (SEQ ID NO:3), IDDKS (SEQ ID NO:4), VIDDKS (SEQ ID NO:5), DDKSG (SEQ ID NO:6), IDDKSG (SEQ ID NO:7), VIDDKSG (SEQ ID NO:8), FVIDDK (SEQ ID NO:9), FVIDDKS (SEQ ID NO:10), FVIDDKSG (SEQ ID NO:11), IFVIDDK (SEQ ID NO:12), IFVIDDKS (SEQ ID NO:13), IFVIDDKSG (SEQ ID NO:14), EEY, IEEY (SEQ ID NO:15), EEYT (SEQ ID NO:16), VIEEY (SEQ ID NO:17), IEEYT (SEQ ID NO:18), VIEEYT (SEQ ID NO:19), EEYTG (SEQ ID NO:20), IEEYTG (SEQ ID NO:21), VIEEYTG (SEQ ID NO:22), FVIEEY (SEQ ID NO:23), FVIEEYT (SEQ ID NO:24), FVEEEYTG (SEQ ID NO:25), FFVIEEY (SEQ ID NO:26), FFVIEEYT (SEQ ID NO:27), FFVEEYTG (SEQ ID NO:28), EAQ, VEAQ (SEQ ID NO:29), EAQT (SEQ ID NO:30), SVEAQ (SEQ ID NO:31), VEAQT (SEQ ID NO:32), SVEAQT (SEQ ID NO:33), EAQTG (SEQ ID NO:34), VEAQTG (SEQ ID NO:35), SVEAQTG (SEQ ID NO:36), FSVEAQ (SEQ ID NO:37), FSVEAQT (SEQ ID NO:38), FSVEAQTG (SEQ ID NO:39), YFSVEAQ (SEQ ID NO:40), YFSVEAQT (SEQ ID NO:41), and YFSVEAQTG (SEQ ID NO:42).

The cadherin polypeptides (and fragments and functionally equivalent fragments thereof) may contain conservative amino acid substitutions. As used herein, “conservative amino acid substitution” refers to an amino acid substitution which does not alter the relative charge or size characteristics of the peptide in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Specific cadherins or cadherin combinations are endogenously expressed on different cell and tissue types, and in different diseases and disorders. This cell-specific cadherin expression profile surprisingly has been found to provide specific “addresses” to which cadherin-expressing cells may be targeted. For example, the presence of a particular cadherin on for example kidney cells allows cells that express a ligand for that cadherin to be targeted to those cells. In some instances, the cells are genetically manipulated include a cadherin molecule that is substantially similar, if not identical, to the cadherin expressed by the target cell or tissue, particularly if homophilic adhesion is desired. As will be understood by one of ordinary skill in the art, the existence of numerous specific and in preferred instances unique addresses on target cells and tissues allows for the production of cells that express the corresponding cadherin addresses.

The compositions and cells of the invention may therefore be selectively targeted to tissues that express one or more cadherin ligands (e.g., cadherin or cadherin receptor) on their surface. The term “cadherin ligand” means a cadherin molecule or a naturally occurring cadherin extracellular domain binding partner that specifically binds to a cadherin molecule.

As used herein, the term “target tissue” or “target cell” means the tissue or cell to which the compositions or cells of the invention are targeted. The target cells or tissues of the invention include tissues that are “normal” (i.e., they are disease- and/or disorder-free). The target cells or tissues of the invention may also be cells and tissues that are “abnormal” (i.e., they are not disease- and/or disorder-free). Abnormal cells or tissues also include cells and tissues that are predisposed to a disease or disorder even though they do not presently manifest disease or disorder characteristics. An example of this latter category is a cell or tissue that harbors a genetic mutation but which has yet to manifest a mutant phenotype. Abnormal cells or tissues are also referred to herein as diseased.

Cadherins each have a particular tissue expression profile. For example, E-cadherin has been reported to be expressed in eye, liver, ovary, pancreas and skin. N-cadherin is reportedly expressed in eye, neural cells, ovary and hematopoietic cells. P-cadherin is reportedly expressed in eye, placenta, peritoneum and endometrial tissue. Cadherin-15/M-cadherin is reportedly expressed in skeletal muscle, satellite cells and cerebellum. Cadherin-11 is reportedly expressed in adipocytes, osteoblasts, synoviocytes, cancer cells and pericytes. Cadherin-5/VE-cadherin is reportedly expressed in endothelial cells. Cadherin-6/K-cadherin is reportedly expressed in embryonic kidney cells. Human mu-protocadherin is reportedly expressed in fetal and adult kidneys. Desmoglein 1 and desmoglein 3 expression has been found in epidermis and keratinocytes. T-cadherin is reportedly expressed in carotid artery wall after balloon angioplasty. Ksp-cadherin is reportedly expressed in developing kidney and genitourinary tract.

Cadherins have been implicated in various diseases and disorders. In these instances, cadherins may play a role in the disorder or may simply be expressed by the cells involved. For example, E-cadherin is expressed in cells involved in cirrhosis, liver injury, burns, papillary thyroid carcinoma, pancreatic adenocarcinoma, peripheral pulmonary adenocarcinomas, and prostate cancer. N-cadherin is expressed in cells involved in Alzheimer's disease and Parkinson's disease. M-cadherin is expressed in cells involved in muscular dystrophy. Cadherin-6/K-cadherin is expressed in cells involved in kidney cancer. P-cadherin is expressed in cells involved in endometriosis.

The invention embraces the use of genetically modified cells expressing cadherin molecules in the treatment of disorders and diseases. In some embodiments, the cells have been genetically modified to express a cadherin that they do not normally express. In other embodiments, the cells are genetically modified to express a cadherin that would normally be expressed by such a cell type.

The genetically modified cells may also express other molecules such as but not limited to reporter markers, screening markers or other therapeutic agents. Reporter markers are markers used to confirm that a nucleic acid of interest has been introduced into a cell. Reporter nucleic acids encode gene products that are either directly or indirectly visualized, and include but are not limited to β-galactosidase, green fluorescent protein, horse radish peroxidase, etc. Screening markers are markers used to select cells that contain the nucleic acid of interest (and conversely to negatively select against cells that lack the nucleic acid of interest) by imparting resistance to a cytotoxic agent. Examples include neomycin (G418), hygromycin, bleomycin, phleomycin and puromycin.

If the targeted cells are intended to repopulate an organ or tissue, and if they are known to be defective in one or more polypeptides, then they can be genetically manipulated to express a wild-type form of the defective polypeptide. Alternatively, they may be manipulated to express a polypeptide that they do not endogenously express but which would be therapeutically beneficial. Such polypeptides include but are not limited to anti-TNF agents (e.g., cytokine receptors such as p55 and p75); anti-IL-1 agents (e.g., cytokine receptors such as IL-1R1 and IL-1R2, and receptor antagonists such as IL-1RA); anti-costimulatory agents (e.g., CTLA-4Ig); anti-inflammatory cytokines (e.g., IL-4 and IL-10); anti-bone resorptive agents (e.g., osteoprotegerin (e.g., OPG, a competitive inhibitor of binding of RANKL to RANK); complement inhibitors (e.g., DAF, and C1 Inh); additional anti-inflammatory agents (e.g., TGF-β); angiogenesis inhibitors (e.g., angiostatin, endostatin, thrombospondin 1, VEGF inhibitors, angiopoeitin inhibitors, and bFGF inhibitors); anti-TGF-β; and anti-cancer agents. For some of the inhibitors recited above, Cross et al. describe soluble receptors and mAbs that are suitable (see Cross, M. J. & L. Claesson-Welsh. Trends Pharmacol Sci 22:201. 2001). These therapeutic agents may be expressed by the cell or attached to the cell surface.

The compositions of the invention useful for delivering cells to target tissues or locations include cells that are genetically modified to express on their surface a cadherin polypeptide comprising at least an extracellular portion of a cadherin or a mutant cadherin. In some embodiments, the cells are genetically modified to express cadherin-containing multi-domain proteins. An example of a multi-domain protein is a fusion protein such as but not limited to a cadherin-Fc fusion protein.

According to one aspect of the invention, cells that are genetically modified to express a cadherin molecule are trafficked in vivo to specific tissues and/or cells that express the counterpart cadherin ligand (e.g., the same cadherin). The cadherin molecule may be a cadherin known to be expressed in a normal target cell, or it may be a cadherin known to be expressed in an abnormal target cell. For example, a cell that is genetically modified to express a normal N-cadherin, when administered to a subject, will specifically target and bind to a cell in the subject that naturally expresses N-cadherin or another N-cadherin ligand. In other embodiments, a cell that is genetically modified to express a mutant form of a cadherin, (e.g., a mutant N-cadherin) on its surface, when administered to a subject, will specifically target a cell that is expressing a wild-type N-cadherin or a substantially similar mutant form of N-cadherin on its surface. Usually, the mutant cadherin preferably will be able to bind to its normal counterpart, but it may lack or include other domains and/or corresponding functions. For example, the mutant cadherin may differ from a wild-type cadherin in the intracellular domain (thereby affecting its signal transduction activity). The mutant cadherin may comprise an intracellular domain from another molecule.

As used herein, the term genetically modified means modifying the normal endogenous cadherin expression in a cell. As used herein, the term “normal expression” means the endogenous level of cadherin expression for that cell type. The modification may result in an increase or decrease in the expression of a normal or mutant cadherin molecule. For example, the cell that is genetically modified may be a cell that does not endogenously express a particular cadherin and thus, genetically modifying the cell to express the cadherin will increase the expression level of cadherin. In another example, a cell may already endogenously express a cadherin, and genetically modifying the cell to express the same cadherin may increase the level of expression of that cadherin. In some embodiments, a cell may endogenously express one type of cadherin, and may be genetically modified to express one or more other types of cadherin. For example, the cell may endogenously express E-cadherin and may be genetically modified to express N-cadherin also. The cadherin expression is cadherin cell surface expression. In preferred embodiments, the amount of the cadherin polypeptide expressed on the cell surface will be an amount sufficient to target the genetically modified cells to desired target tissues. Similarly, for cells of the invention on which a cadherin molecule has been attached by nongenetic methods, the amount of the cadherin polypeptide on the cell surface is sufficient to target the cells to target tissues. A cadherin may be attached to a cell surface by nongenetic methods by ionically attaching a mutant cadherin dimer having two different domains each of which binds to a different cadherin. The mutant cadherin dimer may be attached to a cell having cell surface expression of a cadherin that binds to one domain of the mutant cadherin dimer. The bispecific mutant dimer then has attached to its cell surface a cadherin that it would not otherwise express, and this is accomplished by nongenetic means. In still other embodiments, the cells are genetically modified to reduce or eliminate expression of an endogenously expressed cadherin. Mechanisms for reducing or eliminating endogenous gene expression in a cell are known in the art and include a “knock-out” homologous recombination and antisense technologies. This will alter where the cell will preferentially target. The cell may be administered simply lacking expression of one or more endogenous cadherin polypeptides or the cell may additionally include a recombinant cadherin.

In still another embodiment, a cell may be genetically modified to express a mutant cadherin and such cells may or may not also endogenously express the normal cadherin counterpart. Cells that are genetically modified to express a mutant form of a cadherin may be used to specifically target a cell or tissue in which the mutant form of the cadherin is expressed, e.g., as the result of a disorder or disease such as cancer, but their use is not so limited.

In some aspects of the invention, the genetically modified cells are delivered as a replacement cells or as an additional or supplemental cells to augment a tissue. Examples of tissues that can be targeted in this manner include tissues that have degenerated, that are non-functional, or that are malignant. Tissues known to be affected in these manners include practically all tissues of the body, and in particular neural tissue (e.g., in Alzheimer's or Parkinson's), muscle tissue (e.g., in muscular dystrophy), cardiac cells (e.g. post-infarction), hepatic cells (e.g., in acute hepatic failure due to toxic insult or chronic hepatic failure due to genetic deficiency such as glycogen storage diseases, an example of which is amylo-1,6-glucosidase deficiency), pancreatic islet β-cells (e.g., in diabetes), skin (e.g., to repopulate the skin of burn victims), and bone marrow (e.g., in transplantation). Cadherins that can be used in these methods include, but are not limited to, N-cadherin with neural tissue, M-cadherin with muscle tissue, E-cadherin with liver tissue, and E-cadherin with skin tissue.

In these embodiments, the genetically modified cells may be administered locally or systemically to a subject. The advantage of the cadherin-associated “address” is that the cells can be administered systemically yet still localize and attach to the tissue of interest. Administration routes for the genetically modified cells include but are not limited to parenteral routes such as intravenous administration, intratissue administration, intraperitoneal administration, subcutaneously administration, and the like.

The genetically modified cells may be primary cells. A primary cell is a cell that has been harvested from a subject (but not necessarily the subject being treated). Primary cells can be cultured in vitro prior to infusion into a subject, for example to expand their number. The primary cells may or may not derive from the same tissue or same cell type as the target tissue. For example, in some instances, fibroblasts may be genetically modified and delivered to a non-fibroblast tissue. The cells may be syngeneic, allogeneic or xenogeneic.

The genetically modified cells may also be cells derived from a cell line, such as a human stem cell line. Examples of such lines are described in U.S. Pat. Nos. 6,200,806 and 5,843,780.

In still other embodiments, the genetically modified cells are non-experimental cells. A non-experimental cell is a cell that is not routinely used in in vitro transfection assays. A non-experimental cell also is amenable to infusion into a mammalian subject, preferably a human subject. Some aspects of the invention intend to exclude experimental cells such as the mouse fibroblast cell line NIH-3T3, the human kidney cell lines HEK 293 cells, other cell lines such as BOSC23, and the like.

The genetically modified cells include cells with various proliferative and differentiative potentials. These cells are collectively referred to herein as progenitor cells indicating that they have proliferative and differentiative potential. Thus, the genetically modified cells may be totipotent stem cells which are capable of repopulating an entire organism (i.e., they possess full proliferative and differentiative potential). These cells include embryonic stem cells and the recently identified human stem cells. The genetically modified cells may also be pluripotent stem cells. Pluripotent stem cells are cells that can repopulate one or more tissues or cell types. Generally, pluripotent stem cells repopulate the tissue in which they are located. For example, a hematopoietic stem cell can repopulate the hematopoietic compartment of a subject including repopulation of myeloid and lymphoid cell lineages when it is seeded within a hematopoietic organ or tissue (e.g., the bone marrow, the spleen, and the like). Similarly, a liver stem cell can repopulate the liver of a subject if it is present in the liver. It has recently been reported however that within each of these tissues or organs there exist stem cells that are capable of repopulating other tissues or organs. As a result, it is now possible to repopulate neural tissue using hematopoietic stem cells, and vice versa. Thus, pluripotent stem cells may be tissue-specific or not depending upon their localization in a subject. Examples of pluripotent stem cells include hematopoietic stem cells, liver (hepatic) stem cells, neural stem cells, muscle stem cells, and epidermal (skin) stem cells.

The genetically modified cells may also be multipotent precursor cells. Multipotent precursor cells are cells that have the capacity to differentiate into several cell lineages but which have less proliferative capacity than stem cells. Proliferative capacity refers to the number of cell cycles with the cell can undergo prior to becoming a final end stage proliferatively inert cell. It does not refer to the frequency with which a cell undergoes division, since it is known that most stem cells are quiescent in vivo until they are required for repopulation purposes. Multipotent precursor cells generally differentiate into related cell lineages such as hematopoietic cell lineages or neural cell lineages or liver cell lineages. They do not generally differentiate into cell lineages from disparate organs or tissues. Examples of multipotent precursor cells of the hematopoietic system include CFC-GEMM (capable of differentiating into granulocytes, erythroid, macrophage and megakaryocyte lineages), CFC-GM (capable of differentiating into granulocyte and macrophage lineages), CFC-EMeg (capable of differentiating into erythroid and megakaryocyte lineages), and the like. Similar multipotent precursor cells can be identified in other tissues.

The genetically modified cells may also be unipotent precursor cells. Unipotent precursor cells are cells that are capable of differentiating into only one cell lineage. Unipotent precursor cells have even less proliferative capacity than multipotent precursor cells. Examples of unipotent precursor cells of the hematopoietic system include CFC-M (capable of differentiating into macrophages), CFC-G (capable of differentiating into granulocytes), BFU-E (capable of differentiating into erythroid lineages), and the like. Similar unipotent precursor cells can be identified in other tissues.

The genetically modified cells may also be terminally mature end stage cells. Terminally mature end stage cells are not progenitor cells as used herein. As mentioned above, these cells are proliferatively inert. They are terminally differentiated (i.e., they possess all the functional attributes of their lineage, apart from any proliferative potential). They may also reside in the body for varying periods of time, depending upon the normal lifespan of such cells, and any insult or injury sustained by them or the subject. They may exist for days, weeks and in some cases months. Examples of terminally mature end stage cells of the hematopoietic system include erythrocytes, macrophages, neutrophils, lymphocytes, etc. Other examples include fibroblasts, synoviocytes, hepatocytes, neural cells, and pancreatic islet β-cells. Similar terminally mature end stage cells can be identified in other tissues.

Thus, it is to be understood that the genetically modified cells may be lineage or tissue restricted. The selection of the appropriate cell will depend upon the application. Those of ordinary skill in the art will be able to determine which cells are appropriate for which conditions based on the teachings provided herein and the level of ordinary skill in the art.

The invention is premised in part on the unexpected finding that augmentation of normal or mutant cadherin polypeptides on the surface of cells can alter their ability to target specific cell and/or tissues types. Thus, the invention provides methods for delivering, engrafting or targeting the modified cadherin-expressing cells to specific tissues, and compositions useful therefore.

As used herein, the term “cadherin on the surface of a cell” includes a cadherin molecule present on the surface of a cell that binds a cadherin ligand. A cadherin can be on the surface of the cell as a result of genetic manipulation of the cell, as described herein. Alternatively, the cadherin can be on the surface of the cell as a result of conjugation (e.g., enzymatic or chemical) to the cell surface of proteins expressed thereon. As used herein, a “cadherin-binding portion of a cadherin” is that portion of a cadherin polypeptide that is necessary and sufficient for binding to a cadherin ligand on the surface of a cell, preferably on a distinct and separate cell. Binding portions may include extracellular fragments of cadherins, optionally fused to other polypeptides, and mutated variant cadherin polypeptides that retain the ability to bind cadherins or mutant cadherins, and the like. The cadherin-binding portions of cadherins can be identified by standard methods of molecular biology. For example, extracellular fragments of a cadherin polypeptide can be prepared by deletion of portions of a nucleic acid encoding the cadherin polypeptide using techniques such as PCR, exonuclease digestion, restriction endonuclease digestion to prepare fragments of the nucleic acid molecules, and then cloning the extracellular fragments.

Functionally equivalent variants of cadherin polypeptides can also be used in the inventions. Functionally equivalent variants are defined as peptides or polypeptides with the same qualitative binding characteristics as the parent peptide of polypeptide. The variant may however bind with a different binding constant provided its binding specifically remains unchanged. Substitutions in the amino acid sequence of a cadherin polypeptide to produce functionally equivalent variants of the cadherin polypeptide preferably are conservative substitutions, and typically are made by alteration of a nucleic acid encoding the cadherin polypeptide. Such substitutions can be made by a variety of methods known to one of ordinary skill in the art. For example, amino acid substitutions may be made by PCR-directed mutation, site-directed mutagenesis according to the method of Kunkel (Proc. Natl. Acad. Sci. U.S.A. 82: 488-492, 1985), or by chemical synthesis of a gene encoding a cadherin polypeptide. The activity of functionally equivalent fragments of cadherin polypeptides can be tested by cloning the gene encoding the altered cadherin polypeptide into a bacterial or mammalian expression vector, introducing the vector into an appropriate host cell, expressing the altered cadherin polypeptide, and testing for the functional capability of the cadherin polypeptides as disclosed herein. Such fragments or variants can then be tested for binding to cadherin polypeptides on a cell surface using a variety of assays well known in the art, including capillary flow assays (von Andrian et al., Cell 82:989-999, 1995), parallel plate flow chamber assays (Lawrence and Springer, Cell 65:859-873, 1991; Diacovo et al., Science, 273: 252-255, 1996), intravital microscopy (von Andrian, Microcirculation 3:287-300, 1996), and the like.

As used herein with respect to the modified cells, “expand” means to increase the number of cells in a population by culturing the cells. Preferably the cells are expanded by culturing in vitro under defined culture conditions, such as in medium containing cytokines, growth factors and other nutrients required for the proliferation and/or maturation of the specific cell type to be genetically modified. In some instances, it may be preferable to harvest stem or precursor cells and to maintain them in culture without significant differentiation. In other instance, it may be preferable to harvest stem and precursor cells and culture them to induce their differentiation including potentially terminal differentiation.

“Transfection”, as used herein, refers to the introduction of a plasmid or other non-viral nucleic acid molecule into the target cell. “Transduction”, as used herein, refers to the introduction of the virus genome into the target cell. Transfection includes introduction of naked nucleic acids such as plasmids by standard physical and chemical transfection techniques, including calcium phosphate precipitation, dextran sulfate precipitation, electroporation, liposome-mediated nucleic acid transfer, ballistic methods such as particle bombardment, etc. Transfection also includes introduction of nucleic acids into cells by biological methods, including viral transduction or infection (receptor-mediated and non-receptor-mediated).

In certain embodiments, isolated cells are delivered independently and in other embodiments they are delivered in conjunction with one or more other agents. As used herein, “in conjunction with” means either (1) directly attached to or (2) delivered with but not directly attached to the genetically modified cells. In preferred embodiments, the modified cells are not delivered in conjunction with a soluble cadherin.

As used herein, “isolated” means separated from its native environment and present in sufficient quantity to permit its identification or use. As used herein with respect to cells, isolated means removed from other cell types present in a tissue. For example, the cells may be disaggregated, separated from any capsule or vasculative or blood components, as the case may be. As used herein, “cultured” cells are cells maintained in vitro at 37° C., regardless of whether they undergo division or differentiation during the culture period. Isolated and/or cultured cells preferably are substantially pure, but need not be for the methods and compositions of the invention. Substantially pure populations of cells can be prepared by techniques well known in the art including immunoaffinity purification using chromatography or magnetic separation schemes, gradient density centrifugation, fluorescence activated cell sorting (FACS), and the like. Cells that are produced due to the differentiation of a substantially pure population of stem or precursor cells also are considered substantially pure.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid as used herein is not a naturally occurring chromosome.

As used herein, “isolated” in reference to a protein or polypeptide, means, for example: (i) selectively produced by expression cloning or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

Methods of the invention include, in some aspects, ex vivo therapy. In general, ex vivo therapy involves the introduction in vitro of a nucleic acid that encodes a cadherin into a cell, and administering the genetically modified cell to the subject to deliver the cell to a specific tissue or cell type. The cells may be removed from a subject for in vitro expansion and/or differentiation. Nucleic acids encoding a cadherin polypeptide are introduced (i.e., transduced or transfected) into the cells in vitro. Typically, the modified cells are then expanded in culture before being implanted into a subject. In some embodiments, the original cells are obtained from the subject to whom the genetically modified cells are to be implanted; in other embodiments, the cells may be obtained from a source or person that is not the subject into whom the genetically modified cells are implanted.

As used herein, “subject” means a mammal, including a human, a non-human primate, a horse, a sheep, a pig, a goat, a cow, a dog, a cat, and a rodent. In some embodiments, the preferred subject is a human.

Thus, the invention is not limited in utility to human therapy, but also provides a method for genetically modifying cells in other mammalian subjects such as non-human primates, horses, pigs, sheep, dogs, rodents, and cows. Additionally, the methods and compositions may be used in cultured cells, including long term cultured cells and cell lines.

The compositions and methods of the invention are useful for modulating cadherin activity in a tissue, e.g. by implanting genetically modified cadherin-expressing cells to repopulate a tissue or organ that is otherwise lacking in the cadherin activity or expresses the cadherin at abnormally reduced levels. It is also possible to repopulate tissues or organs that express a cadherin at abnormally increased levels with genetically modified cells that do not express the cadherin or express lower levels of a cadherin. Repopulation of a tissue that expresses a mutant cadherin with cells that express the normal cadherin counterpart is also embraced by the invention. Cadherin activities include cell binding such as homophilic and heterophilic cell binding, secretion of molecules such as, but not limited to, matrix metalloproteinases, stromelysin, collagen, collagenase and cytokines (e.g., IL-6), induction of matrix metalloproteinase (MMP) expression, signal transduction, cell migration, cell invasion, apoptosis, cell proliferation, cell segregation, and cell attachment.

Some of the disorders or diseases sought to be treated using the methods of the invention are cadherin-associated disorders. Cadherin-associated disorders are disorders which at a minimum involve cells that express a cadherin, or cells that abnormally lack cadherin expression. The cadherin molecule may or may not be implicated in the causation or progression of the disorder. Cadherin-associated disorders include, but are not limited to cancer, arthritis, joint inflammation in the synovium, bone and/or joint destruction, inflammatory bowel disease, inflammation of the intestine, nephritis, inflammation of the kidney, vasculitis, inflammation of the blood vessels, asthma, lung inflammation, islet cell inflammation, diabetes-associated inflammation of the pancreas, autoimmune hepatitis, liver inflammation, cryoglobulin-associated serositis, inflammation of the lining of body cavities, fibrosis of the lung, pulmonary fibrosing conditions, fibrosis of the skin, fibrosis of the organs, systemic sclerosis, neurologic disease, neurologic damage, Parkinson's disease, Alzheimer's disease, muscular dystropy, diabetes, cardiac infarction, multiple sclerosis (MS), and skin inflammation (e.g., psoriasis, etc.).

As described above herein, cells express particular cadherin molecules, of which only certain subsets may be expressed in a particular tissue type, e.g. providing an “address” for that particular tissue or cell type. Thus, cells can be prepared that express a cadherin that corresponds to the cadherin type on the specific tissue to be targeted. Cells can be genetically modified to express one or more specific cadherins or variants thereof or mutant cadherins or variants thereof, to reflect the temporal or spatial pattern of cadherin expression of the tissue type to be targeted. Likewise, cells can be genetically modified to express combinations of cadherins to target a plurality of specific cell types.

In some embodiments, the cadherin polypeptide is introduced (e.g., transfected or transduced) into cells by a plasmid vector, non-limiting examples of which include pCEP4 and pDCNA4 vectors. In some embodiments, the cadherin polypeptide is introduced into cells by a viral vector selected from the group consisting of adenoviruses, retroviruses, adeno-associated viruses, poxviruses including vaccinia viruses and attenuated poxviruses, lentiviruses including HIV and HIV-derived viruses, Semliki Forest virus, Venezuelan equine encephalitis virus, Sindbis virus, lambda bacteriophage and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venezuelan equine encephalitis virus (Davis et al., J. Virol. 70:3781-3787, 1996), Sindbis virus (Pugachev et al., Virology 212:587-594, 1995) and modified bacteriophage lambda (PCT/US97/12928, WO96/21007). In some embodiments, the vector is pMIEV. The secondary agents of the invention also may be expressed in the cells using such vectors.

In some embodiments, the viral vectors are replication defective. As used herein, a “replication-defective” virus or viral vector is one which is incapable of replicating autonomously in the target cell. Generally, the genome of a replication-defective adenovirus used in the context of the present invention contains mutations or deletions of at least the sequences needed for replication of the adenovirus in the infected cell. Such sequences are well known to those of ordinary skill in the art, and include, for example, in adenoviruses portions of the E1, E3, and E4 regions of the genome.

In one embodiment, the virus vector is an adenovirus. An adenovirus for the delivery of nucleic acids encoding cadherin polypeptides, refers to an adenovirus that contains exogenous genetic material that can be transcribed and translated in a mammalian cell and which encodes a polypeptide that binds a cadherin on the surface of a cell, i.e., a cadherin polypeptide. The complete nucleotide sequences of adenovirus genomes are known and have been deposited in nucleotide sequence databases. For example, the genome of the adenovirus type 5 has been completely sequenced and is accessible via GenBank accession number M73260. Similarly, portions or even whole genomes of other adenovirus types (type 2, type 7, type 12, and the like), retroviruses, and other viral vectors have also been sequenced and deposited in databases.

The nucleic acid encoding a cadherin polypeptide thereof preferably is inserted into a region of the virus genome that is not essential to the production of replication-defective recombinant viruses. For example, the nucleic acid preferably is not inserted into regions that contain adenovirus genes encoding proteins which are not easily supplied in trans. Thus, the nucleic acid preferably is inserted into the E1 region, which can be complemented (supplied in trans) by an adenovirus encapsidation cell line such as 293 cells. Other preferred sites of insertion of the nucleic acid include the E3 region, which is not required for production of replication-defective recombinant adenoviruses, and the E4 region, mutation of which can be complemented by co-transduction with a helper virus or plasmid or by infection of a suitable complementary cell line. Other sites also may be used as will be apparent to one of ordinary skill in the art. In particular, access to the nucleotide sequences of virus genomes enables a person skilled in the art to identify regions of the virus genomes suitable for insertion of the nucleic acid encoding a cadherin polypeptide.

The nucleic acids assembled to prepare a complete replication-defective virus genome or other viral or non-viral vector can be prepared by any method known in the art. For example, a virus genome or plasmid can be isolated and then modified in vitro by standard methods of molecular biology (see, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York). The modified virus genome so obtained optionally can be isolated and used to transfect an encapsidation cell line if necessary.

In the preferred embodiments, the vector genome further includes a regulatory sequence, e.g., a promoter region (also referred to as a “promoter”), that is operably coupled to the nucleic acid molecule encoding a cadherin or cadherin precursor molecule. The regulatory sequence controls the expression of the nucleic acid molecule encoding a cadherin polypeptide, and/or a secondary agent in the cell. As used herein, a nucleic acid molecule encoding one or more polypeptides (the “coding sequence”) and regulatory sequences are said to be “operably” joined when they are covalently linked in such a way as to place the transcription or the expression of the coding sequence under the influence or control of the regulatory sequences. If it is desired that the coding sequence be translated into a functional protein, two DNA sequences are said to be operably joined if induction of a promoter in the 5′ regulatory sequence results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 3′ or 5′ non-transcribed and non-translated sequences involved with the initiation of transcription and translation respectively, such as a TATA box, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream 5′ or downstream 3′ transcriptional regulatory sequences as desired.

Exemplary promoters that are useful in the invention include constitutive promoters and regulatable promoters (e.g., cell lineage specific promoters, inducible promoters). Exemplary constitutive promoters include promoters derived from cytomegalovirus, a long terminal repeat (LTR) of retroviruses, e.g., Rous sarcoma virus or Moloney murine leukemia virus, and adenovirus E1A promoter, an adenovirus MLP promoter and a SRα promoter. Exemplary tissue or cell specific transcriptional regulatory sequences are those which are active in dendritic cells, including CD11a, dectins-1 and 2, MHC class II, CD1a, b or c, CD80 and CD86. Exemplary inducible promoters are described in the following references: Science 268:1786 (1995); TIBS 18:471 (1993); PNAS 91:3180 (1994); PNAS 90:1657 (1993); PNAS 88:698 (1991); Nature Biotechnol. 14:486 (1996); and PNAS 93:5185 (1996). An exemplary repressible promoter, the tetracycline repressible system, is described in PNAS 89:5547 (1992). Other constitutive, tissue-specific, inducible and repressible promoters will be known by those of skill in the art and thus are not listed here.

Cadherin promoters include the E-cadherin promoter described by Stemmler et al. (Dev. Dyn. 2003 227(2):2380245, the kidney-specific Ksp-cadherin promoter described by Shao et al. (J. Am. Soc. Nephrol. 2002 13(7):1824-36), and the H-cadherin promoter described by Toyooka et al. (Cancer Res. 2002 62(12):3382-6).

The expression vectors (e.g., plasmids, viruses, etc.) optionally contain one or more sequences that are suitable for use in the identification of cells that have or have not been transfected or transduced. Markers to identify cells that have been transfected or transduced include, for example, genes encoding proteins that increase or decrease resistance or sensitivity to antibiotics or other compounds, genes that encode enzymes having activities that are detectable by standard assays known in the art and genes which detectably (e.g. visibly) affect the phenotype of the transduced target cells. Exemplary genes that are suitable as markers include a lacZ gene, a chloramphenicol acetyltransferase gene, an alkaline phosphatase gene, a luciferase gene, and a green fluorescent protein gene. Preferred markers are those which can be used as a basis for selection by fluorescence activated cell sorting or magnetic sorting.

Methods for delivering whole encapsidated virus include contacting cells with the virus, whereby the virus genome can be delivered by receptor-mediated endocytosis via binding of a viral capsid protein to a cellular receptor. Methods for delivering non-encapsidated viral genomes and other nucleic acid molecules such as plasmids include the foregoing methods and also methods for delivery of nucleic acids to cells familiar to those of skill in the art of molecular biology. For example, when delivering a recombinant viral genome without any associated coat protein, or an expression plasmid, the nucleic acid can be introduced into a cell by transfection using a standard technique such as electroporation, liposome transfection, calcium phosphate precipitation, or a commercially available technology such as the Tfx-50 transfection reagent (Promega Corp., Madison, Wis.).

The genetically modified cells of the invention can be delivered to a subject by methods known to those of ordinary skill in the art, particularly parenteral administration methods. Preferably a subject is injected intraarterially, intravenously, intramuscularly, intraperitoneally, or subcutaneously with the modified cells.

The invention provides other compositions and kits which are useful for practicing the above-described methods. According to another aspect of the invention, kits are provided which contain (a) nucleic acid molecules that encodes a cadherin polypeptide; and (b) optionally a nucleic acid encoding a reporter marker, a selection marker, a therapeutic agent, etc. Instructions for the use of the nucleic acid encoding the cadherin polypeptide can also be included. The components of the kits are sufficient, when used, for example, to modify cells isolated from a subject and subsequently administered to a subject, to modulate cadherin activity in the subject, e.g. in the prevention or treatment of a cadherin-associated disorder. In other aspects, the kits may also contain the precursor or undifferentiated cells either in a genetically modified form or not.

The above-described methods and compositions relate to the administration of cells that are genetically modified to express a cadherin and also relate to the delivery of such cells to subjects to prevent, treat, or diagnose cadherin-associated disorders. The methods involve the addition of cadherin-expressing cells to a subject, thus increasing cadherin activity in a given tissue by increasing the amount of cadherin activity present in the tissue. The invention also embraces additional methods of increasing cadherin activity, as well as methods of decreasing cadherin activity.

These methods of the invention involve the modification or alteration of cadherin activity in cells that have endogenous cadherin expression, e.g. the modulation of cadherin activity in cells that are already present in a tissue or subject. Modulation of cadherin activity embraces increasing or decreasing the activity of a tissue, an organ and, in some instances, a cell. It will be understood by those of ordinary skill in the art that an increase in activity includes an increase from zero activity to a level significantly above zero activity as well as an increase from an initial level of activity that is greater than zero activity to a significantly higher level of activity. It will be understood by those of ordinary skill in the art that a decrease in activity includes a decrease from a level above zero activity to a significantly lower level that is also above zero activity as well as a decrease from a level above zero activity to a level of zero activity.

Fusion polypeptides of cadherin are also embraced by the present invention, as is their use in the methods disclosed herein. A fusion polypeptide, as used herein, is a polypeptide that contains peptide regions from at least two different proteins. Fusion proteins include Fc fusions, GST fusions, and the like. A fusion cadherin polypeptide can be formed by fusing, usually at the nucleotide level, coding sequence of a cadherin to coding sequence of another distinct protein. A cadherin-Fc fusion protein can be synthesized by joining the extracellular domains of cadherin to the Fc portion of immunoglobulin. Depending on their nature, some fusion proteins are suited for in vitro use while others are better suited to in vivo use.

The Examples demonstrate a method for synthesizing a cadherin-Fc fusion protein. The invention intends to capture non-cadherin-11 Fc fusion proteins and non-E-cadherin Fc fusion proteins including but not limited to N-cadherin Fc fusion protein, P-cadherin Fc fusion protein, VE-cadherin Fc fusion protein, R-cadherin Fc fusion protein, M-cadherin Fc fusion protein, C-cadherin Fc fusion protein, cadherin-4 Fc fusion protein, cadherin-6 (K-cadherin) Fc fusion protein, cadherin-7 Fc fusion protein, cadherin-8 Fc fusion protein, cadherin-9 Fc fusion protein, cadherin-10 Fc fusion proteins, cadherin-12 (Br-cadherin) Fc fusion protein, cadherin-13 (T- (truncated) cadherin or H- (heart) cadherin) Fc fusion protein, cadherin-14 Fc fusion protein, cadherin-15 (M-cadherin) Fc fusion protein, cadherin-19 Fc fusion protein, cadherin-20 Fc fusion protein, ksp-cadherin Fc fusion protein, PB-cadherin fusion protein, LI-cadherin Fc fusion protein, T-cadherin Fc fusion protein, protocadherin fusion proteins (e.g., protocadherin-42 Fc fusion protein, protocadherin-43 Fc fusion protein, protocadherin-68 Fc fusion protein), protocadherin alpha 1 Fc fusion protein, desmocollin Fc fusion proteins (e.g., desmocollin-1 Fc fusion protein, desmocollin-2 Fc fusion protein, desmocollin-3 Fc fusion protein, desmocollin-4 Fc fusion protein), desmoglein Fc fusion protein (e.g., desmoglein-1 Fc fusion protein, desmoglein-2 Fc fusion protein), protocadherin beta 15 Fc fusion protein, protocadherin gamma A1 Fc fusion protein, protocadherin gamma B1 Fc fusion protein, protocadherin gamma C3 Fc fusion protein, PCDH7 (BH-Pcdh)a Fc fusion protein, protocadherin (PCDH8) Fc fusion protein, protocadherin-Xa Fc fusion protein, and OL-protocadherin Fc fusion protein.

The compositions of the invention include pharmaceutical compositions. The pharmaceutical compositions of the invention may include a composition of the invention in a pharmaceutical acceptable carrier. The pharmaceutical compositions used in the methods should be sterile and contain a therapeutically effective amount of the genetically modified cells or the cadherin molecule compositions.

For therapeutic applications, it is generally that amount sufficient to achieve a medically desirable result. In general, a therapeutically effective amount is that amount necessary to delay the onset of, inhibit the progression of, or halt altogether the particular condition being treated. As an example, the effective amount is generally that amount which serves to alleviate the symptoms (e.g., pain, inflammation, etc.) of the disorders described herein. The effective amount will depend upon the mode of administration, the particular condition being treated and the desired outcome. It will also depend upon the stage of the condition, the severity of the condition, the age and physical condition of the subject being treated, the nature of concurrent therapy, if any, the duration of the treatment, the specific route of administration and like factors within the knowledge and expertise of the medical practitioner. For prophylactic applications, it is that amount sufficient to delay the onset of, inhibit the progression of, or halt altogether the particular condition being prevented, and may be measured by the amount required to prevent the onset of symptoms.

In some aspects, the effective amount is an amount that alone, or together with further doses, increases or decreases the level of cadherin activity as desired. The cadherin activity response can be measured by using methods provided herein, e.g. by determining the level of cadherin activity after the administration of the modified cells, and preferably both before and after the administration of the modified cells. In specific aspects of the invention relating to the targeting of genetically engineered cells, the effective amount is that amount which will cause the cells to localize and/or be maintained in the target tissue.

The preferred amount can be determined by one of ordinary skill in the art in accordance with standard practice for determining optimum dosage levels of the agent. It is generally preferred that a maximum dose of a cadherin-expressing cell that is the highest safe dose according to sound medical judgment be used.

The genetically modified cells of the invention can be administered to a subject in need of such treatment in combination with concurrent therapy for treating the particular disorder or disease the subject is experiencing. The concurrent therapy may be invasive, such as a surgical removal, or may involve drug therapy such as the administration a pharmaceutical preparation. The drug therapies are administered in amounts which are effective to achieve the physiological goals of the specific disorder to be treated, in combination with the genetically modified cells of the invention. Thus, it is contemplated that the drug therapies may be administered in amounts which are not capable of preventing or reducing the physiological consequences of a disorder when the drug therapies are administered alone but which are capable of reducing the consequences when administered in combination with the cells of the invention.

The genetically modified cells of the invention may be administered alone or in combination with the above-described drug therapies as part of a pharmaceutical composition. Such a pharmaceutical composition may include the genetically modified cells in combination with any standard physiologically and/or pharmaceutically acceptable carriers which are known in the art. The compositions should be sterile and contain a therapeutically effective amount of the genetically modified cell in a unit of weight or volume suitable for administration to a patient.

The term “pharmaceutically-acceptable carrier” as used herein means one or more compatible solid or liquid filler, diluents or encapsulating substances which are suitable for administration into a human or other animal. The term “pharmaceutically acceptable” means a non-toxic material that does not interfere with the effectiveness of the biological activity of the active ingredients. Pharmaceutically acceptable further means a non-toxic material that is compatible with a biological system such as a cell, cell culture, tissue, or organism. The term “carrier” denotes an organic or inorganic ingredient, natural or synthetic, with which the active ingredient is combined to facilitate the application. The characteristics of the carrier will depend on the route of administration. The components of the pharmaceutical compositions also are capable of being commingled with the agents of the present invention, and with each other, in a manner such that there is no interaction which would substantially impair the desired pharmaceutical efficacy. The pharmaceutically acceptable carrier must be sterile for in vivo administration. Physiologically and pharmaceutically acceptable carriers include diluents, fillers, salts, buffers, stabilizers, solubilizers, and other materials that are well known in the art.

Compositions suitable for parenteral administration conveniently comprise a sterile aqueous preparation of the cadherin molecules, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend, of course, upon the particular drug selected, the severity of the condition being treated, and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule. In preferred embodiments, the pharmaceutical composition is administered directly to the synovium, synovial fluid or joint capsule by injection preferably with a syringe.

Formulations for use in accordance with the methods of the invention include a syringe containing a genetically modified cell, and a pharmaceutically acceptable carrier that is suitable for injection into the subject.

The pharmaceutical compositions may conveniently be presented in unit dosage form and may be prepared by any of the methods well known in the art of pharmacy. All methods include the step of bringing the cadherin molecules into association with a carrier that constitutes one or more accessory ingredients. In general, the compositions are prepared by uniformly and intimately bringing the cadherin molecules into association with a liquid carrier, a finely divided solid carrier, or both, and then, if necessary, shaping the product. Compositions suitable for oral administration may be presented as discrete units, such as capsules, tablets, lozenges, each containing a predetermined amount of the cadherin molecule. Other compositions include suspensions in aqueous liquids or non-aqueous liquids such as a syrup, elixir or an emulsion.

The invention will be more fully understood by reference to the following examples. These examples, however, are merely intended to illustrate the embodiments of the invention and are not to be construed to limit the scope of the invention. It is also to be understood that the reference figures are illustrative only and are not essential to the enablement of the claimed invention.

### EXAMPLES

### Example 1

Production of Cadherin Fusion Proteins.

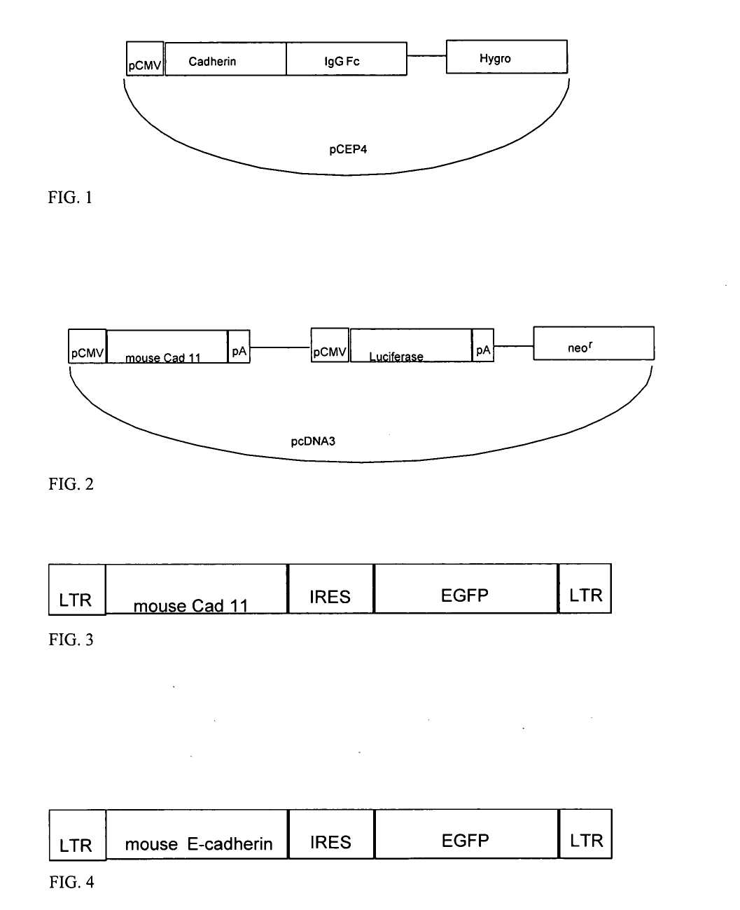

Cadherin-Fc (cad-Fc) fusion proteins (Higgins, J. M., et al., J Cell Biol 140:197, 1998) have been described previously. Mouse cadherins fused to mouse IgG (FIG. 2) are utilized. For human work, human cadherins fused to human IgG (FIG. 3) are used. These cad-fc fusion proteins are produced by mammalian expression systems. The nucleic acid sequence for the extracellular domains of the desired cadherins are placed in front of the Fc domain of the desired IgG molecule. The mammalian expression vector, pCEP4 (Invitrogen Corp., Carlsbad, Calif.), featuring a CMV promoter is used to drive expression of the construct (FIG. 1). These proteins are produced by transfecting mammalian HEK293 cells with this expression vector. Transfection is accomplished with either liposomal methods (such as lipofectamine (Invitrogen Corp.) or via electroporation. Clones are obtained by FACS-based single cell sorting or limiting dilution cloning subsequent to antibiotic selection with hygromycin. High producing transfected clones are identified by an ELISA based screen, assaying for presence of the IgG Fc domain (using anti-species specific IgG conjugated to the HRP enzyme (commercially available from Jackson ImmunoResearch Laboratories, West Grove, Pa.) and subsequent PAGE-western blot biochemical analysis. Fusion proteins are affinity purified from culture supernatants using protein-G sepharose beads (Amersham Pharmacia Biotech, Piscataway, N.J.).

### Example 2

Production and Administration of Cells to be Targeted via Cadherins.

Directed cell trafficking is examined in mice using mouse a cadherin (such as cadherin 11) and an identifiable marker (either the enzyme luciferase or the fluorescent protein GFP) co-transfected into mouse L-cell fibroblasts and/or ex-vivo synovial fibroblasts (see: Han, Z., et al., Arthritis Rheum 46:818. 2002). Stable cadherin-11 expressing L-cell fibroblasts are obtained by transfection (either electroporation or lipofectamine (Invitrogen) (liposomal) method) and antibiotic selection. Antibiotic resistant clones are screened for cadherin 11 expression by western blot assay using rabbit anti-cadherin-11 antisera (Zymed Laboratories, South San Francisco, Calif.) and anti-rabbit HRP secondary development (The Jackson Laboratory, Bar Harbor, Me.). Identifiable marker presence in transfected cells/clones is confirmed by luciferase assay or by visual inspection of cells using fluorescence microscopy (GFP marker). For the GFP variants, GFP expression is quantified using FACS.

To examine directed trafficking, a mouse model of inflammatory arthritis (Korganow, A. S., et al., Immunity 10:451. 1999; Lee, D. M., et al., Science 297:1689. 2002) is used. Cadherin-11 or control transfected, labeled cells are injected into mice with inflammatory arthritis at D=7. 48 hours later, mice are sacrificed and tissues harvested, including, ankles (inflamed joint tissue), skin, liver, spleen, stomach. For experiments done with the enzymatic marker luciferase, these tissues are homogenized with a polytron homogenizer and standard luciferase assays are performed (Ow, D. W., et al., Science 234:856. 1986; de Wet, J. R., et al., Mol Cell Biol 7:725. 1987). Luciferase enzyme activity (quantitative) is assayed as a measure of cellular trafficking to target tissues. For experiments done with the fluorescent protein GFP, tissues are cryopreserved for cryosectioning and histologic analysis. Additionally, for GFP containing experiments, tissues are disaggregated by mincing and collagenase digestion and GFP containing cells are enumerated via FACS.

For these experiments, the cadherin (e.g. cadherin-11) is transfected with the luciferase or GFP marker. This is accomplished in several ways, examples of which are provided below herein.

1) This can be accomplished via co-transfection of independent expression plasmids for each protein. In specific, the pCEP4 vector (Invitrogen) containing mouse cadherin 11 (hygromycin selection) and the pCDNA3 vector (Invitrogen) containing luciferase (G418 selection) are used.

2) Stable double transfectants are obtained by resistance to both G418 and hygromycin transfection of an vector containing both cadherin 11 and luciferase. A dual-promoter vector is constructed in the pCDNA3 (FIG. 2). For this construct, stable transfectants are obtained by G418 selection and subsequent verification of cadherin 11 expression (western blot assay)

3) Viral transduction with a bicistronic (IRES-containing) vector featuring mouse cadherin 11 linked to EGFP are generated. (Hawley ref: Leung, B. L., et al., J Immunol 163:1334. 1999, see FIG. 3).

### Example 3

Delivery of Cells Using Cadherin Targeting.

This method includes use of cadherin targeting to deliver cells to specific tissue locations, for example, delivering stem cells to specific anatomic locations. The cadherins useful in these methods include classical and non-classical cadherins. The entire sequence of cadherin molecule may be used to target specific tissues and the specific combinations of cadherins and cell types to be targeted allows delivery of cells to targeted tissues. For example, a “tissue-appropriate cadherin” can be used to deliver cells to a particular tissue type/location. The use of homophilic adhesion to direct trafficking, means the tissue cadherin is matched to that transfected into the cell (e.g. a stem cell) for delivery. The directed cells, e.g. embryonic stem cells are retained at the targeted anatomic site. Mouse stem cells are transfected with tissue-appropriate cadherins and a traceable marker (GFP, Lac-Z, luciferase) using the transfection method described herein.

A disease model utilizes the fumarylacetoacetate hydrolase (FAH) deficient mouse strain (Grompe, M., M. et al., Genes Dev 7:2298. 1993). This strain experiences progressive liver damage that is fatal unless treated with the chemical NTBC. This model allows a regulated state of chronic hepatic damage; and the damage provides a signal for recruitment of regenerative stem cells. Previous experiments have shown that exogenous hematopoietic (bone marrow) stem cells are capable of transdifferentiating into hepatocytes with regenerative function in these mice (Lagasse, E., H. et al., Nat Med 6:1229. 2000). Because hepatocytes express E-cadherin, mock-cadherin, GFP transfected and E-cadherin, GFP transfected ES cells are introduced into FAH-deficient mice and the ability of these ES cells to regenerate damaged liver is quantified. Stable ES cells from the 129 mouse strain are utilized. These cells are transfected by: electroporation, liposomal method (lipofectamine, above), or via viral transduction. For viral transduction, the IRES-containing vectors are utilized for bicistronic expression of two cDNAs off of one mRNA transcript. For this experiment, mouse E-cadherin is used in the 5′ MCS and GFP is used in the 3′ MCS, and the viral vector MIEV is used. The presence of GFP expression provides a quantifiable marker of ES cell progeny presence utilizing histologic, flow cytometric and biochemical techniques. Twelve weeks are allowed for engraftment, proliferation and differentiation of the transferred ES cells. Thereafter, mice are sacrificed and liver, spleen, stomach, skin and tongue tissues are cryopreserved, cryosectioned and assessed for GFP presence within the tissues. Presence of GFP signifies contribution of the donor ES cells to the hepatic parenchyma. To demonstrate a quantitative increase of hepatic contribution from E-cadherin transfected ES cells, an increase hepatic GFP is determined relative to mock transfected ES cells.

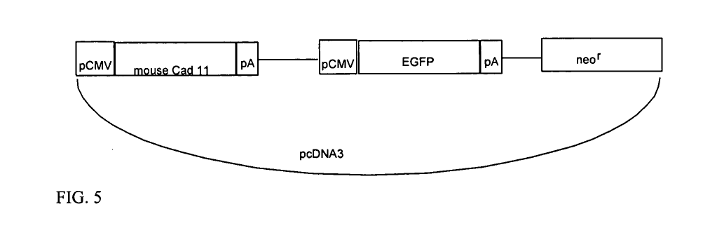

As an alternative to the FAH deficient mouse model, cadherin- (e.g. cadherin-11) regulated trafficking of transfected ES cells to cadherin 11 expressing inflamed mouse synovium is assessed. Cadherin-11 expression has previously been demonstrated on arthritic mouse synovial tissues. In this experimental series, inflammatory arthritis is induced using the KRN serum transfer model developed by Diane Mathis et al. (Korganow, A. S., et al., Immunity 10:451. 1999). Utilizing arthritogenic serum from KRN transgenic mice allows a rapid, reproducible stimulus for development of arthritis in susceptible mouse strains. To demonstrate a cadherin-dependent (e.g. cadherin 11-dependent) increase in ES cell contribution to inflamed synovial tissue, mock and cadherin transfected (e.g. cadherin 11 transfected) GFP-expressing ES cells are injected into mice with inflammatory arthritis (for constructs, see mCad11pGFP pcDNA3 (FIG. 5) or mCad11-pMIEV (FIG. 4). ES cell contribution to synovial tissue is quantified by utilizing histologic and biochemical (western blot) enumeration of GFP as an indication of cellular trafficking.

Various types of stem cells are used in these methods. For mice standard ES cell lines are used. These require standard, well-defined culture and transfection conditions (Robertson, E. J. Teratocarcinomas and Embryonic Stem Cells: A Practical Approach. IRL Press. 1987.). Human cells are isolated and cultured using standard procedures, such as Thomson, J. A., et al., Science 282:1145. 1998 (for review see Amit, M., & J. Itskovitz-Eldor. J Anat 200:225. 2002).

Mouse bone marrow stromal stem cells and human bone marrow cells from donors are also used in the methods described. Technically, this is accomplished as for the studies with mouse ES cells above but with the modification that bone marrow mesenchymal stem cells (see Jiang, Y., et al., Nature 418:41. 2002) are transduced with mCAD11-pMIEV (GFP expressing virus).

Temporal Expression Control Methods

Stem cell cadherin expression is controlled temporally and spatially in some instances. For example, in some modifications transient transfectants are used so the cadherin is only expressed initially and the construct is “diluted out” in daughter cells. This reduces concern for insertion-related cellular transformation for use in humans, but is not essential for use of the methods.

For application of the methods in humans, embryonic stem cells and other stem cell compartments are also utilized. In mice, bone marrow stem cells are used (e.g. bone marrow stromal (mesenchymal) stem cells and or hematopoeitic vs stromal (mesenchymal). These method are the same as described above herein for the FAH mouse model, except input cells are ex-vivo bone marrow stem cells (see Jiang, Y., et al., Nature 418:41. 2002).

The cells for delivery are injected intravenously for delivery and localize via the vascular system to their targets.

### Example 4

Delivery of Organ Specific Cells to Replace Damaged Tissues.

Delivery of cells to replace damaged tissues is done using methods described in Example 2. Organ-specific cells are generated using the various methods. For example, disaggregated pancreatic β-cells are transplanted into diabetic mice (made diabetic via chemical streptozosin (Phelan, S. A., et al., Diabetes 46:1189. 1997). In this method a donor pancreas is disaggregated and β-cells isolated as described (Esni, F., et al., J Cell Biol 144:325. 1999). These cells are transduced with E-cadherin/GFP or control empty/GFP containing viral construct (pMIEV) and injected IV into recipient diabetic mice. To assess clinical effectiveness of transplanted islet cells, serum glucose is monitored for a period of 4 weeks. Mice are sacrificed and liver, skin, splenic, stomach and tongue tissues are harvested and cryosectioned. Islet cell implantation from E-cadherin transfectants relative to control transfectants is assessed by GFP content of recipient tissues. Human cells are isolated and transferred using standard methods for human β-cell isolation and transfer as described in Shapiro, A. M., et al., N Engl J Med 343:230. 2000.

The above-described method is also used with β-cells differentiated from ES cells (Lumelsky, N., et al., Science 292.1389. 2001) instead of the disaggregated pancreatic β-cells.

Disorders for which the cadherin-targeted cells are administered include neurological injury, stroke, Parkinson's disease, Alzheimer's disease, Fulminant failure due to toxicity, Chronic failure due to hepatitis or alcohol abuse. the cadherin-targeted cells are also used for cardiac myocyte regeneration after infarction, pancreatic islet cell regeneration in diabetes, myocyte replacement in muscular dystrophy, and hepatic regeneration. Additionally, the methods are used for to correct metabolic deficiencies, such as glycogen storage diseases, for example, Amylo-1,6-glucosidase deficiency.

For these prevention and treatment methods, the bone marrow stem cell protocol using FAH deficient bone marrow (as described above) is used. The methods are those described above for the FAH mouse model, except input cells are ex-vivo bone marrow stem cells (see: Jiang, Y., et al., Nature 418:41. 2002). Included in the cadherin viral vector is the correct cDNA for the FAH enzyme to get coordinate expression of E-cadherin and FAH. These bone marrow stem cells are re-introduced via intravenous (IV) injection.

### References

1. Cepek, K. L., S. K. Shaw, C. M. Parker, G. J. Russell, J. S. Morrow, D. L. Rimm, and M. B. Brenner. 1994. Adhesion between epithelial cells and T lymphocytes mediated by E-cadherin and the alpha E beta 7 integrin. Nature 372:190.
2. Cross, M. J., and L. Claesson-Welsh. 2001. FGF and VEGF function in angiogenesis: signalling pathways, biological responses and therapeutic inhibition. Trends Pharmacol Sci 22:201.
3. Kreitman, R. J., R. K. Puri, P. Leland, B. Lee, and I. Pastan. 1994. Site-specific conjugation to interleukin 4 containing mutated cysteine residues produces interleukin 4-toxin conjugates with improved binding and activity. Biochemistry 33:11637.
4. Senter, P. D., and C. J. Springer. 2001. Selective activation of anticancer prodrugs by monoclonal antibody-enzyme conjugates. Adv Drug Deliv Rev 53:247.
5. Higgins, J. M., D. A. Mandlebrot, S. K. Shaw, G. J. Russell, E. A. Murphy, Y. T. Chen, W. J. Nelson, C. M. Parker, and M. B. Brenner. 1998. Direct and regulated interaction of integrin alphaEbeta7 with E-cadherin. J Cell Biol 140:197.
6. Korganow, A. S., H. Ji, S. Mangialaio, V. Duchatelle, R. Pelanda, T. Martin, C. Degott, H. Kikutani, K. Rajewsky, J. L. Pasquali, C. Benoist, and D. Mathis. 1999. From systemic T cell self-reactivity to organ-specific autoimmune disease via immunoglobulins. Immunity 10:451.
7. Lee, D. M., D. S. Friend, M. F. Gurish, C. Benoist, D. Mathis, and M. B. Brenner. 2002. Mast cells: a cellular link between autoantibodies and inflammatory arthritis. Science 297:1689.
8. Han, Z., L. Chang, Y. Yamanishi, M. Karin, and G. S. Firestein. 2002. Joint damage and inflammation in c-Jun N-terminal kinase 2 knockout mice with passive murine collagen-induced arthritis. Arthritis Rheum 46:818.
9. Ow, D. W., K. V. Wood, M. DeLuca, J. R. D. Wet, D. R. Helinski, and S. H. Howell. 1986. Transient and Stable Expression of the Firefly Luciferase Gene in Plant Cells and Transgenic Plants. Science 234:856.
10. de Wet, J. R., K. V. Wood, M. DeLuca, D. R. Helinski, and S. Subramani. 1987. Firefly luciferase gene: structure and expression in mammalian cells. Mol Cell Biol 7:725.
11. Leung, B. L., L. Haughn, A. Veillette, R. G. Hawley, R. Rottapel, and M. Julius. 1999. TCR alpha beta-independent CD28 signaling and costimulation require non-CD4-associated Lck. J Immunol 163:1334.
12. Grompe, M., M. al-Dhalimy, M. Finegold, C. N. Ou, T. Burlingame, N. G. Kennaway, and P. Soriano. 1993. Loss of fumarylacetoacetate hydrolase is responsible for the neonatal hepatic dysfunction phenotype of lethal albino mice. Genes Dev 7:2298.
13. Lagasse, E., H. Connors, M. Al-Dhalimy, M. Reitsma, M. Dohse, L. Osborne, X. Wang, M. Finegold, I. L. Weissman, and M. Grompe. 2000. Purified hematopoietic stem cells can differentiate into hepatocytes in vivo. Nat Med 6:1229.
14. Thomson, J. A., J. Itskovitz-Eldor, S. S. Shapiro, M. A. Waknitz, J. J. Swiergiel, V. S. Marshall, and J. M. Jones. 1998. Embryonic stem cell lines derived from human blastocysts. Science 282:1145.
15. Amit, M., and J. Itskovitz-Eldor. 2002. Derivation and spontaneous differentiation of human embryonic stem cells. J Anat 200:225.
16. Jiang, Y., B. N. Jahagirdar, R. L. Reinhardt, R. E. Schwartz, C. D. Keene, X. R. Ortiz-Gonzalez, M. Reyes, T. Lenvik, T. Lund, M. Blackstad, J. Du, S. Aldrich, A. Lisberg, W. C. Low, D. A. Largaespada, and C. M. Verfaillie. 2002. Pluripotency of mesenchymal stem cells derived from adult marrow. Nature 418:41.
17. Phelan, S. A., M. Ito, and M. R. Loeken. 1997. Neural tube defects in embryos of diabetic mice: role of the Pax-3 gene and apoptosis. Diabetes 46:1189.
18. Esni, F., I. B. Taljedal, A. K. Perl, H. Cremer, G. Christofori, and H. Semb. 1999. Neural cell adhesion molecule (N-CAM) is required for cell type segregation and normal ultrastructure in pancreatic islets. J Cell Biol 144:325.
19. Shapiro, A. M., J. R. Lakey, E. A. Ryan, G. S. Korbutt, E. Toth, G. L. Warnock, N. M. Kneteman, and R. V. Rajotte. 2000. Islet transplantation in seven patients with type 1 diabetes mellitus using a glucocorticoid-free immunosuppressive regimen. N Engl J Med 343:230.
20. Lumelsky, N., O. Blondel, P. Laeng, I. Velasco, R. Ravin, and R. McKay. 2001. Differentiation of embryonic stem cells to insulin-secreting structures similar to pancreatic islets. Science 292:1389.
21. Vleminckx, K., L. Vakaet, Jr., M. Mareel, W. Fiers, and F. van Roy. 1991. Genetic manipulation of E-cadherin expression by epithelial tumor cells reveals an invasion suppressor role. Cell 66:107.
22. Kyte, J., and R. F. Doolittle. 1982. A simple method for displaying the hydropathic character of a protein. J Mol Biol 157:105.

### Equivalents

It should be understood that the preceding is merely a detailed description of certain preferred embodiments. It therefore should be apparent to those of ordinary skill in the art that various modifications and equivalents can be made without departing from the spirit and scope of the invention. It is intended that the invention encompass all such modifications within the scope of the appended claims.

All references, patents and patent applications and publications that are recited in this application are incorporated in their entirety herein by reference.

1. A method for delivering cells to a target tissue of a subject, comprising
administering an isolated cell with genetically altered cadherin expression to a subject in need thereof, wherein the genetically altered cadherin expression allows the isolated cell to bind to a target tissue.

2. The method of claim 1, wherein the genetically altered cadherin expression is a decrease in the expression of an endogenously expressed cadherin.

3. The method of claim 1, wherein the genetically altered cadherin expression is an increase in the expression of an endogenously expressed cadherin.

4. The method of claim 1, wherein the genetically altered cadherin expression is expression of a cadherin not endogenously expressed by the isolated cell.

5. The method of claim 1, wherein the genetically altered cadherin expression is lack of expression of an endogenously expressed cadherin.

6. The method of claim 1, wherein the genetically altered cadherin expression is altered expression of two or more cadherins.

7. The method of claim 1, wherein the genetically altered cadherin expression is altered expression of a classical or non-classical cadherin.

8. The method of claim 7, wherein the classical cadherin is selected from the group consisting of E-(epithelial) cadherin, N-(neural) cadherin, P-(placental) cadherin, VE-(vascular endothelial) cadherin, R-(retinal) cadherin, M-cadherin, and C-cadherin.

9. The method of claim 7, wherein the non-classical cadherin is selected from the group consisting of cadherin-6 (K-cadherin), cadherin-7, cadherin-8, cadherin-11 (OB-cadherin), cadherin-12 (Br-cadherin), cadherin-13 (T-(truncated) cadherin or H-(heart) cadherin), cadherin-14, cadherin-15 (M-cadherin), PB-cadherin, LI-cadherin, T-cadherin, protocadherins (e.g., protocadherin-42, protocadherin-43, protocadherin-68), desmocollins (e.g., desmocollin-1, desmocollin-2, desmocollin-3, desmocollin-4), desmogleins (e.g., desmoglein-1, desmoglein-2), and cadherin-related neuronal receptors.

10. The method of claim 1, wherein the genetically altered cadherin expression is expression of a cadherin full length polypeptide or a fragment thereof.

11. The method of claim 1, wherein the genetically altered cadherin expression is expression of a cadherin mutant or a functionally equivalent cadherin variant.

12. The method of claim 1, wherein the isolated cell is a stem cell.

13. The method of claim 12, wherein the stem cell is a totipotent stem cell.

14. The method of claim 13, wherein the totipotent stem cell is an embryonic stem cell.

15. The method of claim 12, wherein the stem cell is a pluripotent stem cell.

16. The method of claim 15, wherein the pluripotent stem cell is tissue-specific.

17. The method of claim 15, wherein the pluripotent stem cell is a neural stem cell, a skin stem cell, a liver stem cell, a pancreatic β islet cell, a mesenchymal stem cell, or a hematopoietic stem cell.

18. The method of claim 1, wherein the isolated cell is a multipotent precursor cell.

19. The method of claim 1, wherein the isolated cell is a unipotent precursor cell.

20. The method of claim 1, wherein the isolated cell is a terminally mature cell.

21. The method of claim 20, wherein the terminally mature cell is selected from the group consisting of a bone marrow stromal cell, a hepatocyte, a synovial cell, a muscle cell, a cardiac cell, a neural cell, and a skin cell.

22. The method of claim 1, wherein the isolated cell is a primary cell.

23. The method of claim 22, wherein the primary cell is harvested from the subject.

24. The method of claim 22, wherein the primary cell is harvested from a different tissue than the target tissue.

25. The method of claim 22, wherein the primary cell is cultured in vitro.

26. The method of claim 1, wherein the isolated cell is derived from a cell line.

27. The method of claim 1, wherein the isolated cell has proliferative activity.

28. The method of claim 1, wherein the isolated cell is administered to the subject parenterally.

29. The method of claim 1, wherein the isolated cell is administered to the subject by intravenous, intraperitoneal, subcutaneous, intramuscular or intratissue administration.

30. The method of claim 1, wherein the cadherin is known to bind to a tissue selected from the group consisting of synovium, blood vessel, lung, bone marrow, bone, colon, kidney, epidermis, joint, brain, neuronal, muscle, pancreas, liver, heart tissue, and pancreas.

31. The method of claim 1, wherein the target tissue is abnormal.

32. The method of claim 1, wherein the subject has or is at risk of developing muscular dystrophy, cirrhosis, a hematopoietic abnormality, Alzheimer's disease, Parkinson's disease, cystic fibrosis, arthritis, nephritis, vasculitis, asthma, autoimmune hepatitis, fibrosis, stroke, diabetes, cardiac infarction, hepatic failure, multiple sclerosis, or cancer.

33. The method of claim 1, wherein the isolated cell has genetically altered reporter marker expression.

34. The method of claim 33, wherein the reporter marker is selected from the group consisting of luciferase, green fluorescent protein (GFP), and β galactosidase.

35. The method of claim 1, wherein the isolated cell has genetically altered selection marker expression.

36. The method of claim 35, wherein the selection marker is selected from the group consisting of neomycin (G418), hygromycin, bleomycin, phleomycin and puromycin.

37. The method of claim 1, wherein genetically altered cadherin expression is transient.

38. The method of claim 1, wherein the cadherin is N-cadherin and the target tissue is neural tissue.

39. The method of claim 1, wherein the cadherin is M-cadherin and the target tissue is muscle.

40. The method of claim 1, wherein the cadherin is E-cadherin and the target tissue is liver or skin.

41. A composition comprising an isolated non-experimental cell having genetically altered cadherin expression.

42-78. (Cancelled)

79. A cell comprising a human progenitor cells genetically altered to express a cadherin.

80.-88. (Cancelled)


SEQUENCE LISTING (42 sequences)

SEQ ID NO: 1 - protein, 4 aa, Homo sapiens
IDDK

SEQ ID NO: 2 - protein, 4 aa, Homo sapiens
DDKS

SEQ ID NO: 3 - protein, 5 aa, Homo sapiens
VIDDK

SEQ ID NO: 4 - protein, 5 aa, Homo sapiens
IDDKS

SEQ ID NO: 5 - protein, 6 aa, Homo sapiens
VIDDKS

SEQ ID NO: 6 - protein, 5 aa, Homo sapiens
DDKSG

SEQ ID NO: 7 - protein, 6 aa, Homo sapiens
IDDKSG

SEQ ID NO: 8 - protein, 7 aa, Homo sapiens
VIDDKSG

SEQ ID NO: 9 - protein, 6 aa, Homo sapiens
FVIDDK

SEQ ID NO: 10 - protein, 7 aa, Homo sapiens
FVIDDKS

SEQ ID NO: 11 - protein, 8 aa, Homo sapiens
FVIDDKSG

SEQ ID NO: 12 - protein, 7 aa, Homo sapiens
IFVIDDK

SEQ ID NO: 13 - protein, 8 aa, Homo sapiens
IFVIDDKS

SEQ ID NO: 14 - protein, 9 aa, Homo sapiens
IFVIDDKSG

SEQ ID NO: 15 - protein, 4 aa, Homo sapiens
IEEY

SEQ ID NO: 16 - protein, 4 aa, Homo sapiens
EEYT

SEQ ID NO: 17 - protein, 5 aa, Homo sapiens
VIEEY

SEQ ID NO: 18 - protein, 5 aa, Homo sapiens
IEEYT

SEQ ID NO: 19 - protein, 6 aa, Homo sapiens
VIEEYT

SEQ ID NO: 20 - protein, 5 aa, Homo sapiens
EEYTG

SEQ ID NO: 21 - protein, 6 aa, Homo sapiens
IEEYTG

SEQ ID NO: 22 - protein, 7 aa, Homo sapiens
VIEEYTG

SEQ ID NO: 23 - protein, 6 aa, Homo sapiens
FVIEEY

SEQ ID NO: 24 - protein, 7 aa, Homo sapiens
FVIEEYT

SEQ ID NO: 25 - protein, 8 aa, Homo sapiens
FVEEEYTG

SEQ ID NO: 26 - protein, 7 aa, Homo sapiens
FFVIEEY

SEQ ID NO: 27 - protein, 8 aa, Homo sapiens
FFVIEEYT

SEQ ID NO: 28 - protein, 8 aa, Homo sapiens
FFVEEYTG

SEQ ID NO: 29 - protein, 4 aa, Homo sapiens
VEAQ

SEQ ID NO: 30 - protein, 4 aa, Homo sapiens
EAQT

SEQ ID NO: 31 - protein, 5 aa, Homo sapiens
SVEAQ

SEQ ID NO: 32 - protein, 5 aa, Homo sapiens
VEAQT

SEQ ID NO: 33 - protein, 6 aa, Homo sapiens
SVEAQT

SEQ ID NO: 34 - protein, 5 aa, Homo sapiens
EAQTG

SEQ ID NO: 35 - protein, 6 aa, Homo sapiens
VEAQTG

SEQ ID NO: 36 - protein, 7 aa, Homo sapiens
SVEAQTG

SEQ ID NO: 37 - protein, 6 aa, Homo sapiens
FSVEAQ

SEQ ID NO: 38 - protein, 7 aa, Homo sapiens
FSVEAQT

SEQ ID NO: 39 - protein, 8 aa, Homo sapiens
FSVEAQTG

SEQ ID NO: 40 - protein, 7 aa, Homo sapiens
YFSVEAQ

SEQ ID NO: 41 - protein, 8 aa, Homo sapiens
YFSVEAQT

SEQ ID NO: 42 - protein, 9 aa, Homo sapiens
YFSVEAQTG